(19) United States
(12) Patent Application Publication
(10) Pub. No.: US 2024/0268698 A1
(43) Pub. Date: Aug. 15, 2024

(54) APPARATUS AND METHOD FOR SURGICAL MARGIN ASSESSMENT USING BIOIMPEDANCE SENSING ARRAY

(71) Applicant: The Trustees of Dartmouth College, Hanover, NH (US)

(72) Inventors: ALLAIRE DOUSSAN, WILDER, VT (US); RYAN HALTER, LYME, NH (US); ETHAN MURPHY, WHITE RIVER JUNCTION, VT (US)

(21) Appl. No.: 18/568,940

(22) PCT Filed: Jun. 10, 2022
(86) PCT No.: PCT/US22/33064

§ 371 Date: Dec. 11, 2023

Related U.S. Application Data
(60) Provisional application No. 63/210,038, filed on Jun. 13, 2021.
(60) Provisional application No. 63/210,126, filed on Jun. 14, 2021.

(51) Int. Cl. A61B 5/0536; A61B 5/00

(52) U.S. Cl. CPC A61B 5/0536; A61B 5/6843; A61B 5/7264

(57) ABSTRACT

A bioimpedance device for surgical cavities has pick-up electrodes surrounded by driving electrodes, each driving electrode having greater area than the pick-up electrodes, the electrode array coupled through an adjacent connector to an electronics module. The electronics module fits through laparoscopic ports. The electronics module includes a voltage controlled current source, multiplexing for driving electrodes, a force sensor, and voltage buffers, and couples to a data acquisition system coupled to a processor. The processor uses the apparatus to perform bioimpedance mapping of tissue. A method of mapping bioimpedance includes contacting tissue with the electrode array, sequentially driving at least one of the driving electrodes with an alternating current at frequencies between 100 and 1000000 hertz while reading the sense electrodes through analog to digital converters into the processor; using readings of the sense electrodes to generate a bioimpedance map of the tissue; the electrode array fitting through laparoscopic ports.

### PRIORITY CLAIM

The present document claims priority to U.S. Provisional patent applications 63/210,126 filed Jun. 14, 2021, and 63/210,038 filed Jun. 13, 2021. The entire contents of both provisional patent applications are incorporated herein by reference.

### GOVERNMENT RIGHTS

This invention was made with government support under grant nos. 5R01CA143020 and 1R01CA237654 awarded by National Institutes of Health, and grant number R41CA235994 awarded by Small Business Technology Transfer (STTR). The government has certain rights in the invention.

### BACKGROUND

There are many types of lesions treatable with surgical removal or modification. These lesions include abnormal tissues in any location in the body, such as malignant (or cancerous) tumors, and many slower growing “benign” tumors. While lesions are of many kinds, it is generally desirable for a surgeon to be able to visualize the lesion being treated and to be able to discriminate between lesion tissues and adjacent normal tissues.

Generally, surgeons treat lesions that are visible to them during surgery. At times, lesions and tumors may lie under the surface of an organ, or under a visible and exposed surface of an operative site, where they may be obscured by overlying tissue and not readily visible or may have poor contrast relative to surrounding tissues. It is desirable to make these lesions, including portions of malignant tumors, detectable to a surgeon so that they can be more readily treated and so the surgeon can limit damage to normal nearby tissue.

Cancer cells of a tumor that originated in an organ often have a superficial appearance that resembles normal tissue of the same organ, giving poor contrast of tumor relative to surrounding tissue.

Bioimpedance is a property related to a tissue's resistance to electrical current flow and its ability to store electrical charge. Bioimpedance is predominantly a function of tissue architecture including cellular size and density, cellular spacing, and the constituents of the extracellular matrix (ECM). Impedance differences between tissue types may be markers for pathologic processes such as cancer. Some investigators report that a tissue's impedance signature may be more sensitive to the presence of tumor tissue than conventional imaging techniques of computed tomography (CT) and ultrasound. Use of electrical impedance signatures to differentiate normal and neoplastic states has been reported in studies of cervical, breast, skin, and bladder tissues.

Tissue bioimpedance, Z, can be determined by applying an alternating current (AC), I, between two electrodes, the driving electrodes, and measuring the resulting potential difference, V, between a second pair of electrodes, the sense electrodes. The AC version of Ohms law, V=IZ, can be used to relate V and I to the bioimpedance of the tissue sample influenced by the electric field established between the voltage measurement or sense electrodes. Complex bioimpedance, Z, combines resistive (R) and reactive (X) components, Z=R+jX, where j is the imaginary number. Alternatively, one can express a complex admittance, Y, consisting of a conductance (G) and capacitance (C) such that Y=G+jωC where ω is the radian frequency (ω=2πf). Admittance is the reciprocal of impedance, =1/Z, and G and C are related to R and X as

$$G = \frac{R}{{R}^{2} + {X}^{2}} \qquad ( 1 )$$

$$and$$

$$C = \frac{- X}{ω ( {R}^{2} + {X}^{2} )} . \qquad ( 2 )$$

R, X, G, and C are parameters that depend on the configuration and geometry of the measurement probe used to make the impedance recordings. Conductivity, σ, and relative permittivity, εᵣ, are related to G and C, but are geometry-independent parameters that are typically used to report impedance properties. In general, σ describes a tissue's ability to allow electric current to flow and has the units of S/m, while εᵣ describes a tissue's relative charge storage capability and is unitless. Conductivity is related to conductance as

$$σ = BG \qquad ( 3 )$$

where B is a geometry factor associated with the probe configuration. Likewise, the relationship between relative permittivity and capacitance is

$${ε}_{r} = BC / {ε}_{o} \qquad ( 4 )$$

where ε₀ is the permittivity in free space, 8.85×10⁻¹² F/m. In the well-known case of a parallel plate geometry, B=d/A, where d and A are the distance between the two plates and their area, respectively. For different spatial arrangements, the geometry factor can be determined analytically or experimentally. An experimental technique involves measuring the impedance of a known load such as de-ionized (DI) water (εᵣ=80) and calculating B using Eq. 4.

A pathological metric used to rate the success of surgical cancer-removal procedures is post-operative evaluation of the surgical margin of the resected tissue. This typically involves cutting the tissue into sections, staining them, and microscopically exploring the sections for the presence of cancer cells at margins of the resected tissue. Cancer cells noted at the margins suggest that cancer cells were left in the body following the procedure and thus represent Positive Surgical Margins (PSMs). Not surprisingly, patients with PSMs have a much higher rate of disease recurrence than patients having negative surgical margins.

Patients with PSMs are often exposed to noxious additional procedures to eradicate the cancer cells left behind including radiation, chemical, hormonal, and additional surgical therapy; these all have adverse morbidities that decrease the patient's quality of life.

### SUMMARY

A bioimpedance device for surgical cavities has pick-up electrodes surrounded by driving electrodes, each driving electrode having greater area than the pick-up electrodes, the electrode array coupled through an adjacent connector to an electronics module. The electronics module fits through laparoscopic ports. The electronics module includes a voltage controlled current source, multiplexing for driving electrodes, a force sensor, and voltage buffers, and couples to a data acquisition system coupled to a processor. The processor uses the apparatus to perform bioimpedance mapping of tissue. A method of mapping bioimpedance includes contacting tissue with the electrode array, sequentially driving at least one of the driving electrodes with an alternating current at frequencies between 100 and 1000000 hertz while reading the sense electrodes through analog to digital converters into the processor; using readings of the sense electrodes to generate a bioimpedance map of the tissue; the electrode array fitting through laparoscopic ports.

### BRIEF DESCRIPTION OF THE FIGURES

FIG. 1 is a diagram illustrating electrodes of a multi-electrode bioimpedance sensing array having 4 driven and 25 voltage-pickup electrodes.

FIG. 2 is a diagram illustrating electrodes of a multi-electrode bioimpedance sensing array having 8 driven and 25 voltage-pickup electrodes in an 8.5 mm diameter probe.

FIG. 3 is a diagram illustrating electrodes of a multi-electrode bioimpedance sensing array having 8 driven and 25 voltage pickup electrodes in a 9.5 mm diameter probe.

FIG. 4 is a block diagram of the data acquisition system used with the multi-electrode bioimpedance sensing arrays of FIGS. 1, 2, and 3.

FIG. 5 is a side view of an electrode connector plug.

FIG. 6 is a side view of an electrode connector socket.

FIG. 7 is a top view of the embodiment of FIG. 1 superimposed on the electrode connector plug to show correspondence of plug connections to electrodes.

FIG. 7A is an overview of an operating room configuration showing where the multi-electrode bioimpedance sensing device fits into an operating room environment.

FIG. 8 is a block diagram of the multi-electrode bioimpedance sensing device.

FIG. 9 is a flowchart of procedures using the probe.

FIG. 10 is a block diagram of a system including the probe.

FIG. 11 is another block diagram of the DAQ and probe.

### DETAILED DESCRIPTION OF THE EMBODIMENTS

Reducing positive surgical margin (PSM) rates through real-time intraoperative assessment of surgical margin status would increase the patient's quality of life by reducing the need for the noxious additional therapies associated with PSMs. Additionally, intraoperative assessment of the margins would allow the surgeon to save healthy tissue by avoiding the need to blindly take larger margins to prevent PSMs. This is especially important for maintaining organ function. For example, nerve-sparing radical prostatectomies have been shown to decrease post-surgical complications such as incontinence and erectile dysfunction which is correlated with an increase in the patient's quality of life.

We have shown that the electrical impedance of tissue is sensitive to the cellular arrangement and can be used to distinguish cancer from benign tissue in the prostate. To this end we have developed multiple prototypes of a flexible endoscopic device capable of sensing the electrical impedance of tissue during radical prostatectomy (RP) and other surgical procedures. This device makes focal measurements of intraoperative margin status which allows real-time diagnostics of PSMs via Machine Learning-based tissue classification, Electrical Impedance Tomography (EIT), and/or Electrical Impedance Spectroscopy techniques. Our electrical impedance probes can be deployed either laparoscopically, such as robotic assisted radical prostatectomy, or in open procedures such as breast conserving surgery, to provide an accurate method of intraoperatively identifying PSMs. When used laparoscopically, the probe 801 is inserted through a laparoscopy port implanted in a patient's abdominal wall into the patient's abdominal cavity where it may contact tissue within the patient.

Our technology has 6 main components. These include:
1) Disposable Probe 801 Tip or Electrode Array 802 with Specialized Electrodes

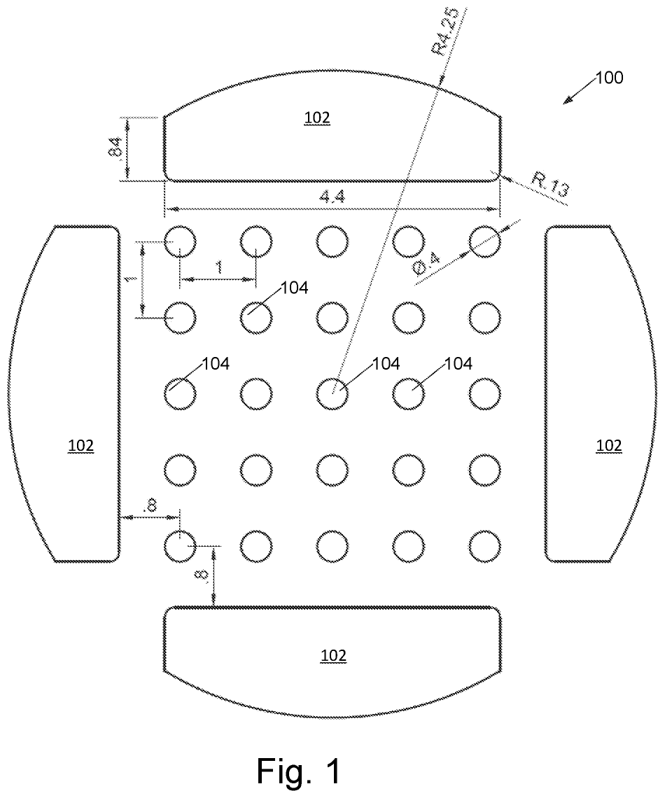

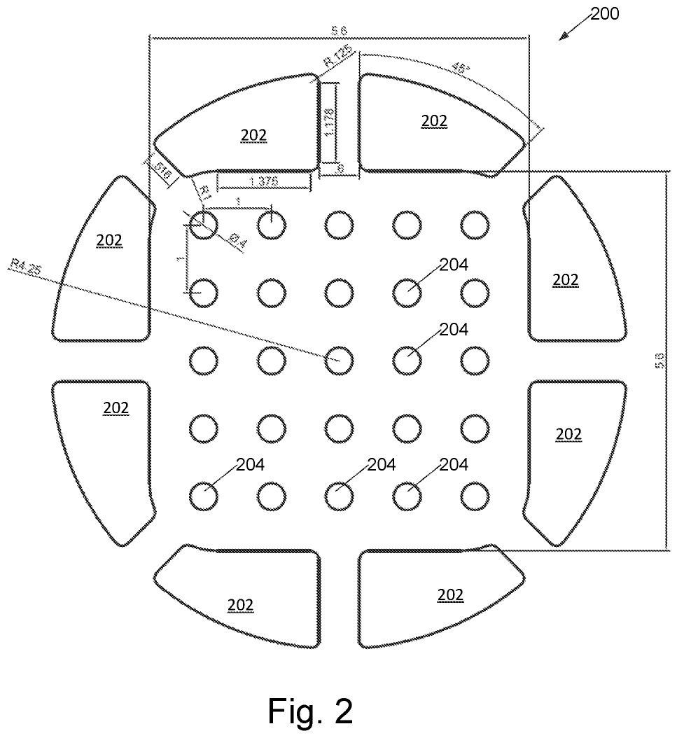

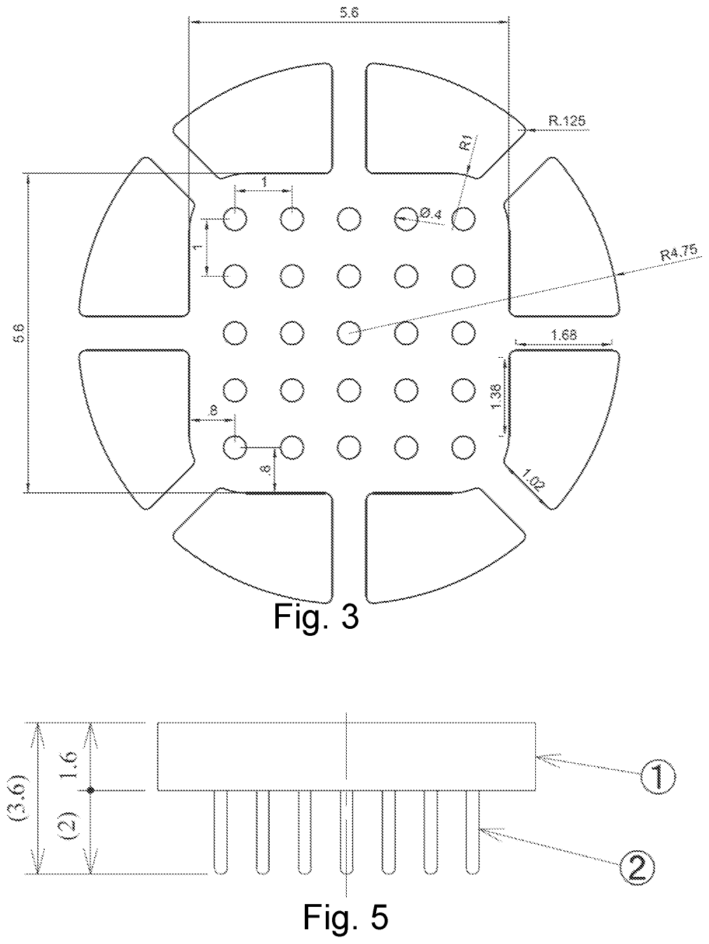

The disposable probe tip or electrode array 802 is replaced for each patient and contains two types of electrodes: current drive electrodes and voltage pick-up electrodes. Our electrode array has four to eight current drive electrodes with a larger area than, and surrounding, twenty-five smaller voltage pick up, or voltage sensing, electrodes. FIGS. 1, 2, and 3 shows versions of our electrode array with various current drive electrode configurations and 0.1256 mm² voltage pick-up electrodes. In the embodiment 100 of FIG. 1, four driving electrodes 102 surround an array of 25 sense electrodes 104. In the embodiment 200 of FIG. 2, eight driving electrodes 202 surround an array of 25 sense electrodes 204. In alternative embodiments there may be as few as sixteen sense electrodes, or there may be more, in a particular embodiment 36, sense electrodes. Each measurement is recorded using a four-probe scheme where two current drive electrodes are used to drive a known current through the tissue and two voltage pickup electrodes are used to measure an induced voltage potential. The four-probe scheme avoids including the contact impedance that typically arises at the electrode-tissue interface of the current drive electrodes in voltage measurements. Because contact impedance is inversely proportional with the electrode size, we use large-area electrodes as the current drive electrodes to reduce the contact impedance and thus loads on the current source (see section below on probe signal conditioning circuitry). Current sources are generally limited to smaller loads; thus, this improves the performance and bandwidth of our system. Another factor in the specialization of electrodes is spatial resolution because smaller area electrodes for voltage pick-up improve the spatial resolution. Our voltage pick-up electrodes are connected through high input impedance voltage buffers which minimizes current flow and voltage drop at electrode-tissue interfaces. Because of this, we do not expect contact impedance to produce significant voltage drops. This allows the voltage pick-up electrodes to be much smaller than the current-drive electrodes, enhancing the spatial resolution of the system which allows for the identification of smaller areas of cancerous tissue.

### 2) Probe Analog Front End (AFE)

The AFE includes signal conditioning circuitry and cabling that connects to the data acquisition (DAQ) system. The signal conditioning circuitry of this probe fits within the probe housing near the probe tip and electrode array allowing for short traces from electrodes to the signal conditioning circuitry. The signal conditioning circuitry reduces the effects of noise on the system as the signals are transmitted back to the Data Acquisition (DAQ) system over a significant length of cabling (on the order of 1 meter), this provides less signal degradation than if the AFE is located at the distal DAQ system instead of being adjacent to the electrode transducer. The signal conditioning circuitry includes a low pass filter followed by an enhanced Howland voltage controlled current source (VCCS) 850 (FIG. 10) with a buffer in its negative feedback loop to convert the voltage waveform produced from the DAQ system into a constant current waveform. The waveform produced is sinusoidal, or a superposition of sinusoids, with frequency components between 100 Hz to 1 MHz. In embodiments, impedance measurements are obtained at multiple frequencies in the range 100 Hz to 1 MHz, and in an alternative embodiment at multiple frequencies in the range 100 Hz to 80 MHz. The signal conditioning circuitry also includes an instrumentation amplifier to monitor the current injected into the patient, source multiplexors 856 and sink multiplexors 858 to select source and sink (current drive) of the driving electrodes 860 in use at any given time, DC blocking capacitors 862 in series with the driving electrodes 860 to isolate the patient, and voltage buffers 852 on signals from the voltage pick-up or sense electrodes 854 to reduce the impact of noise as it travels back to the DAQ system to be sampled and stored (i.e. representing an active electrode design). Additionally, we include a force sensor 816 positioned under the electrode array 802 to measure pressure applied to the probe by a user during a measurement. The measured impedance varies with pressure applied to the tissue, so including a force sensor allows us to take measurements in an optimal pressure range and normalize the impedance measurements based on the force applied to the probe tip. This improves the precision of our technology by removing some user variability. A version with four driving electrodes and 25-channel voltage pick-up or sense electrodes has been tested. A version with eight driving electrodes and 25-voltage pick-up or sense electrodes has been found to provide better resolution than the four driving electrode version. Additional embodiments with twelve or sixteen driving electrodes are expected to also be functional. In anticipated embodiments, the sense electrodes are disposed in a rectangular sense electrode array with the driving electrodes forming a square or circular arrangement around the sense electrode array.

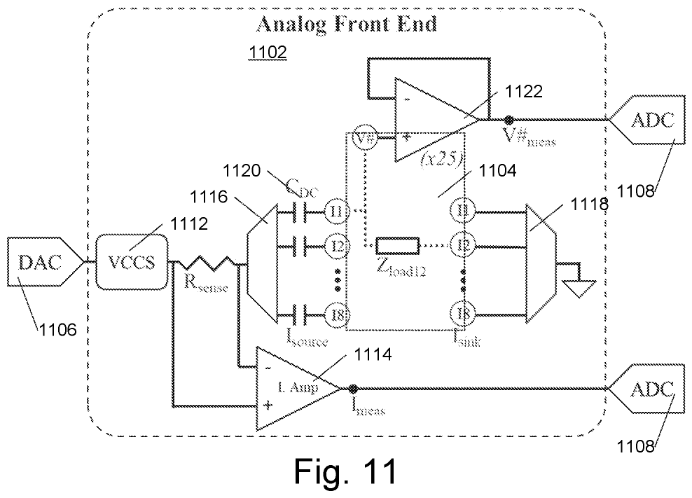

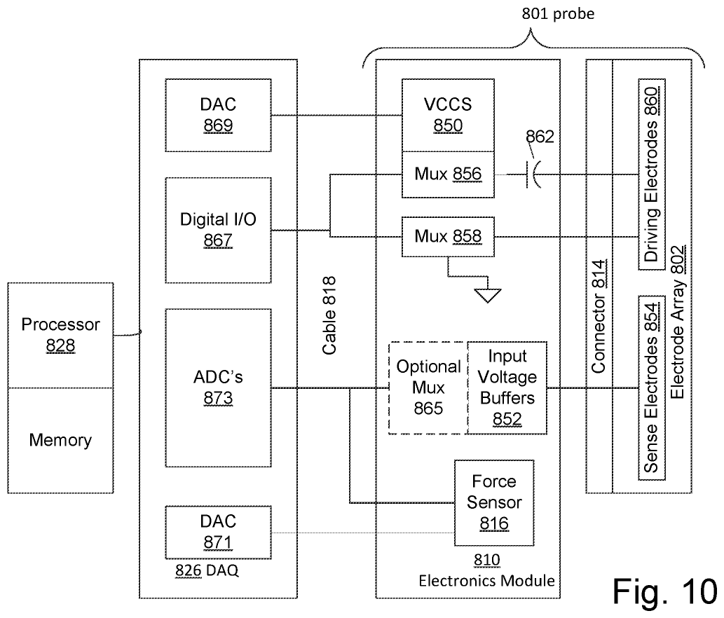

FIG. 11 is another format of block diagram of DAQ with probe 1102 and tissue 1104 showing DAQ blocks DAC 1106 and ADCs 1108, 1110 outside the dotted line; the VCCS 1112, a current sensing circuit 1114, driving multiplexor 1116, ground or current sink multiplexor 1118, DC blocking capacitors 1120, driving electrodes I1 to I8, sense electrodes V #, and buffer amplifiers 1122.

### 3) DAQ System to Sample, Store, and Process the Collected Data

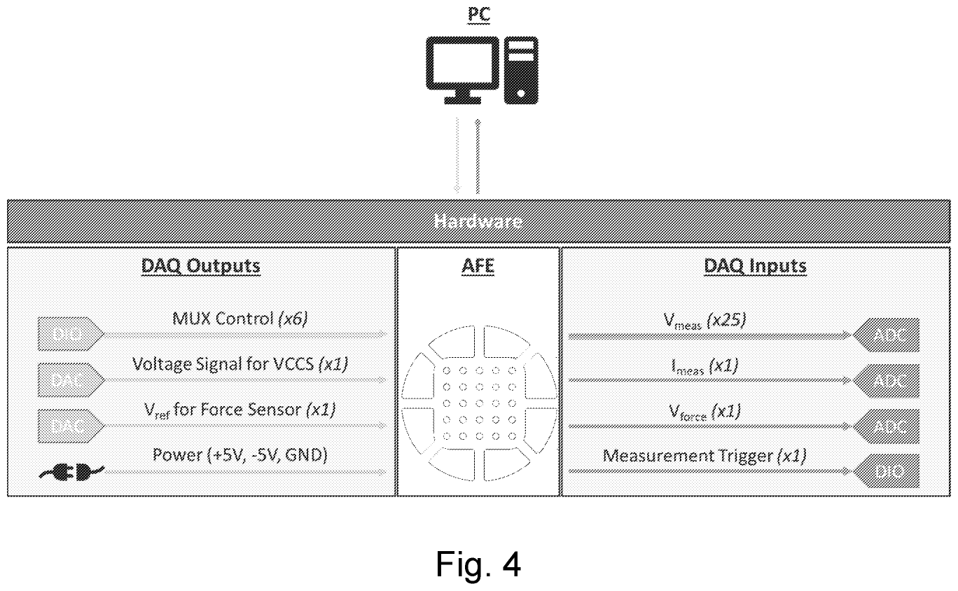

As shown in FIGS. 4, and 10 the DAQ-system 826 has a digital input/output (DIO) unit 867 providing 6 to 8, and in embodiments with an input analog multiplexor 865 up to 16, digital outputs to the AFE include to control the multiplexors 856, 858, 865, a high-resolution waveform generator that may include a 16-bit digital to analog convertor (DAC) 869 to provide input to the VCCS 850 and thus provide stimulus through multiplexors 856, 858, to selected driving electrodes 860, a lower resolution DAC 871 for a force sensor 816 reference voltage, and power supply connections to power the AFE (not shown). The inputs, to the DAQ system, from the AFE, include buffered signals from the 25 voltage sense electrodes 854 to analog-to-digital convertors (ADCs) 873, in an embodiment the ADCs 873 are 14-bit ADCs, one for each of the 25 voltage pick-up measurement channels (Vₘₑₐₛ). There are also analog inputs for a current sense (Iₘₑₐₛ) associated with the VCCS 850, and one for the force sensor 816 (V_{force}) along with an additional DIO that in embodiments may be used for a measurement trigger device to initiate the data acquisition such as a foot pedal or button. The DAQ system then interfaces with a computer (PC) having a processor where digital data corresponding to voltages on the 25 voltage sense electrodes is stored and processed before being displayed to the user. In an alternative embodiment, an analog multiplexor 865 multiplexes the analog inputs from the sense electrodes 854 to a single 14-bit ADC 873 channel. In embodiments where the waveform generator provides a superposition of several sinusoidal waveforms of different frequencies to selected driving electrodes 860, the processor can perform a fast-Fourier transform on the digital data corresponding to voltages on the 25 sense electrodes to separate voltage and phase contributions from each of the several sinusoidal waveforms and determine impedance at each frequency before mapping the impedances.

### 4) Probe Tracking System

In some embodiments, in order to automatically overlay information on the surgeon's console during either robotic, such as those performed using the da Vinci Surgical System, trademark of Intuitive Surgical, Sunnyvale, California or during open procedures using some display or augmented reality, the position and orientation of the probe in space must be known. This can be done with many methods including optical, electromagnetic, or stereovision trackers. Knowing the location of the probe in space allows us to track where each impedance measurement is taken to allow for automatic registration of the measurement results with the correct anatomical location. This reduces the cognitive load for the surgeon as it eliminates the need for mental registration of the results with the intraoperative location on the patient. In some embodiments a tracking unit 824 is mounted in an operating room and tracking transponders 822A are attached to the probe and the patient.

### 5) Data Processing/Classification Algorithm(s)

Potential data processing/classification methods include but are not limited to electrical impedance tomography, electrical impedance spectroscopy, and machine learning based methods. Once clinical data has been collected with the newest version of the probe, we will have a better understanding of what scheme has the best performance for the application.

### 6) User Interface

Depending on the application, the device might have a different user interface. For example, for robotic assisted surgeries using the da Vinci Surgical System (Intuitive Surgical) the ideal user interface might be integrated into the surgeon's console using TilePro (an integrated visualization platform included with the daVinci system). However, for open surgeries this console does not exist and thus the best option might be to use augmented reality to overlay the results on the patient's anatomy. A simpler version might be to display the results to the surgeon via a screen instead. However, for each application the user interface should include the following information: the pressure applied (to find an ideal pressure range prior to taking a measurement), a trigger to start acquiring data, a notification that the data collection has finished, and some method of displaying the results to the surgeon.

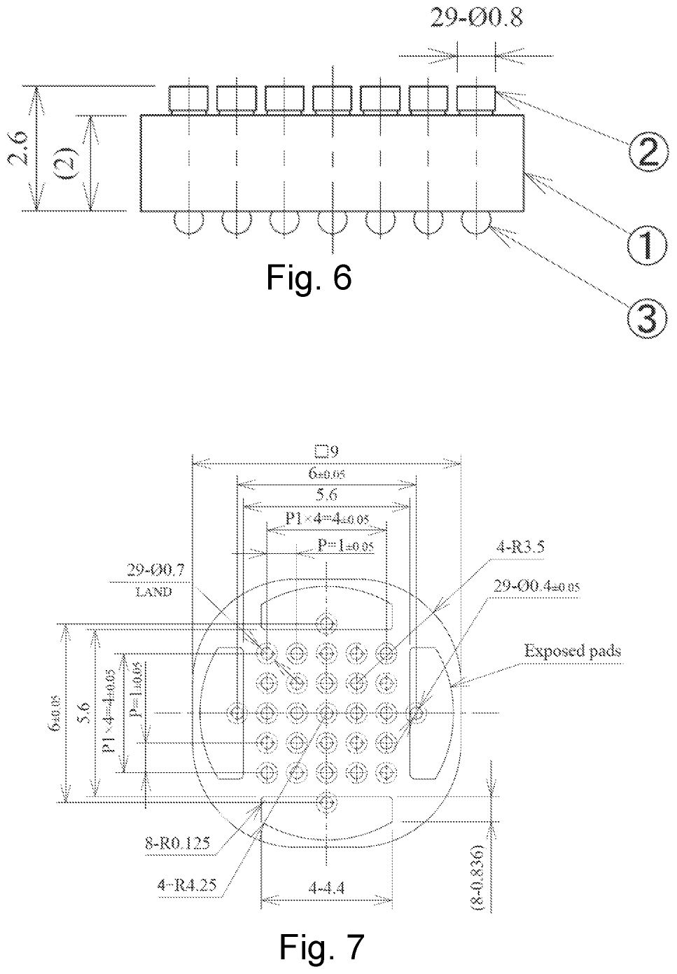

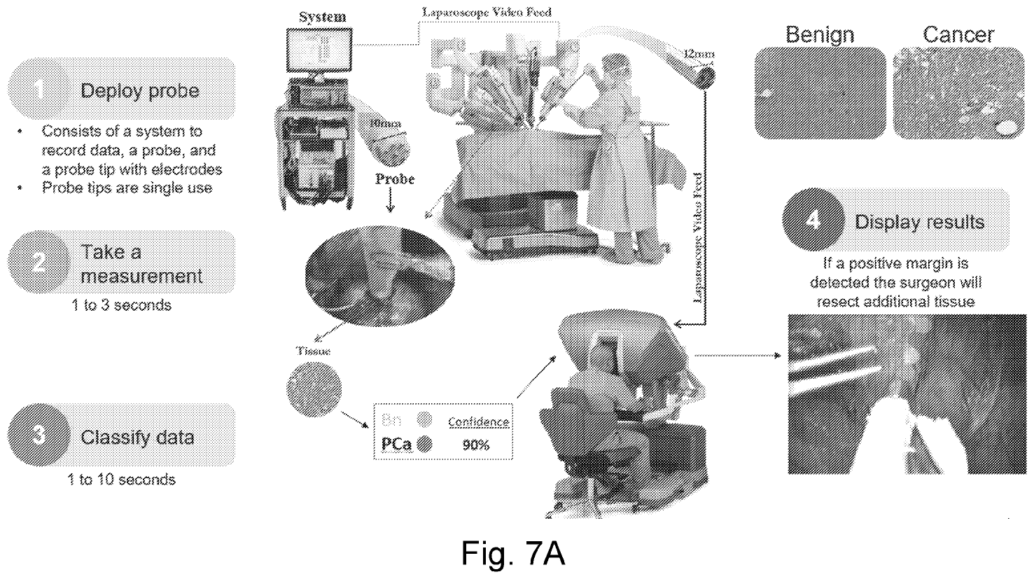

Our embodiment incorporates the following aspects:
1) Electrode array: To our knowledge, no other technologies have electrodes specialized based on their function as described above.
2) Electrode connector: We have an electrode connector 814 that allows the 29 or 33 electrode array to completely detach from the main housing of the probe. The electrode connector plug has gold plated electrodes on the top that contact the patient (FIGS. 1, 2, 3, 7). The plug (FIG. 5) has 29 pins that extend down from each of the electrodes to connect with the electrode connector socket (FIG. 6). The corresponding socket has openings for the pins of the electrode connector plug to fit into and is mounted in the main housing of the probe. The electrode array 802 plug is disposable and used on a per-patient basis while the socket is reusable.
3) Pressure sensing: We incorporate a force sensor 816 to simultaneously record the applied pressure to the probe tip and its electrode array 802 either prior to or during measurements.
4) Active electrode: To reduce the impact of noise on the system, we have moved signal conditioning circuitry such as input voltage buffers 852 to near the tip of the probe, the probe being implemented with a combination of flex and rigid circuit board assembly. This is the smallest 29-channel or 33-channel active electrode system that we are aware of as it is on the order of 12 mm diameter and 5 cm in length.
5) Compatibility with robotic procedures: Our technology was designed to fit within a 12 mm laparoscopic port commonly used in robotic assisted surgical procedures and may also be used to take measurements manually directly from a surgical cavity.
6) Probe housing: Depending on the application we expect to have different probe housings with the same internal contents. We anticipate there may be one version for robotic procedures to interface with the da Vinci robotic system (trademark of Intuitive Surgical, Sunnyvale, California) and one for open procedures that fits ergonomically in a surgeon's hand.
7) Data processing methods: We will develop additional data processing methods once we have collected enough clinical data.
8) Integrated tracking: Existing technology requires manual registration of the results, typically from excised tissue, to the patient's intraoperative margin. By incorporating a tracking system into the probe, we can automatically register the results to the patient's anatomy reducing the computation load for the surgeon.

We provide real-time results that allow the surgeon to identify PSMs intraoperatively and thus these PSMs can be addressed during surgery by resecting the detected cancerous tissue, eliminating the need for additional noxious therapies. It is also non-destructive, so it is safe to use near critical structures such as blood vessels and nervous tissue. This is particularly important in nerve sparing procedures, as there is a conflict between cutting less to prevent impairing normal functions and cutting more tissue out to prevent PSM. We expect that our probe will help the surgeon make data-based decisions about what is best for the patient in these scenarios because the cancerous tissue is not always immediately obvious to the naked eye. Additionally, our probe is compatible with both laparoscopic and open procedures, takes measurements directly in the patient's body cavity instead of on excised tissue, and is non-ionizing. In summary, the electrical impedance probe described here is a versatile device.

In embodiments, channel-to-channel variation between the 4 current injection channels and 25 voltage channels is compensated for with calibration data obtained by recalibrating on a regular basis.

Impedance may change with factors other than tissue properties such as temperature and pressure. We have included a force sensor in the probe and compensate measurements by normalizing the measured impedances with the applied pressure. We expect that the temperature of the operating room will stay constant enough that it will not significantly affect the accuracy of the probe.

Our initial target market is prostate cancer surgeries, which is the treatment for about one third of prostate cancer cases or 69,143 patients in the US per year in 2017. There is the potential to expand this to more solid tumor cancers, which extends our patient population to approximately 1.1 million in the U.S. when considering the 10 most common solid tumor cancers: breast, prostate, bladder, colon and rectum, oral cavity, lung and bronchus, kidney and renal pelvis, uterine, and ovarian cancers. Other embodiments of the rigid probe housing may be used depending on the application, such as open or laparoscopic surgeries, but the contents of the probe and functionality remain the same.

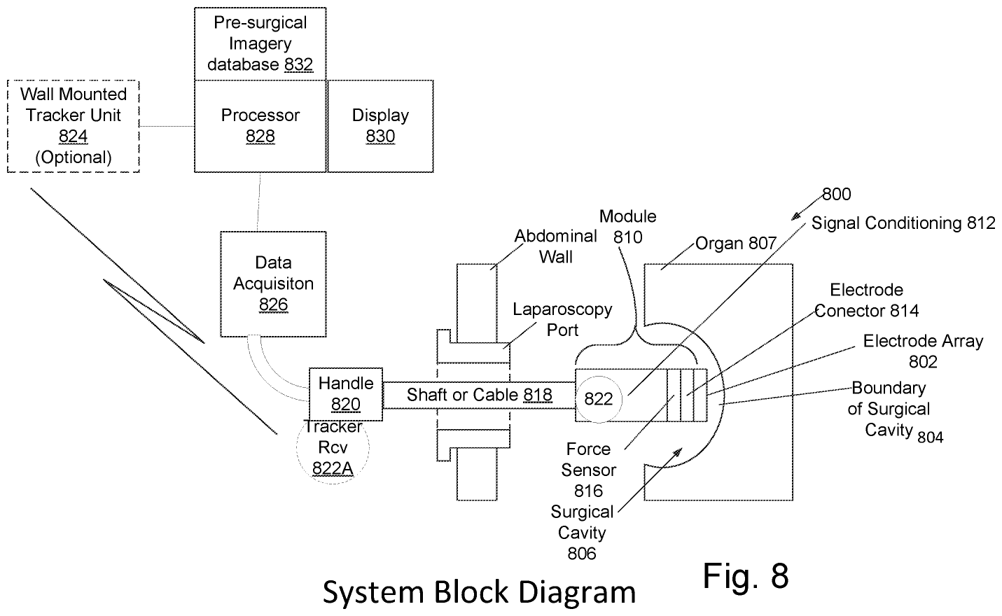

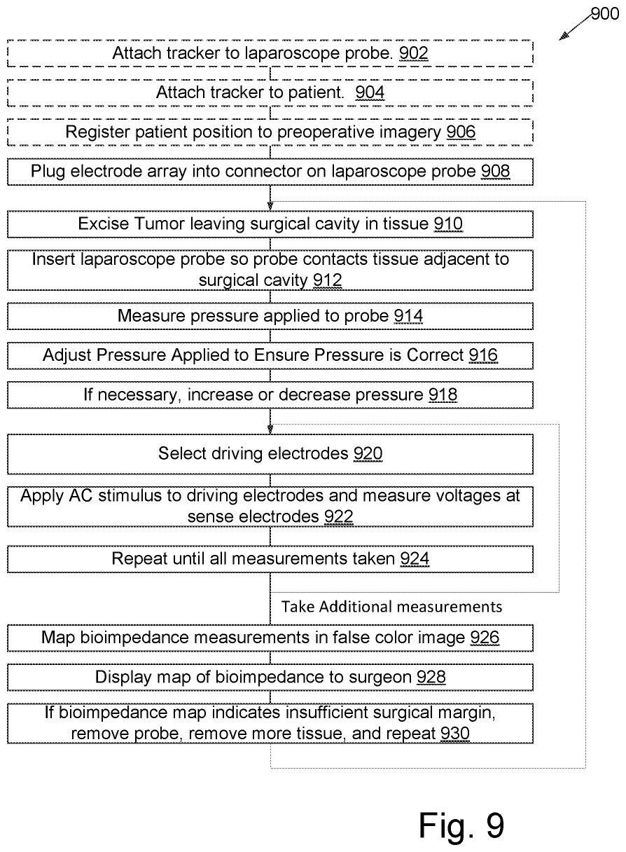

A system 800 (FIG. 8) incorporating the above-described probe and electrode array 802 is used to evaluate a boundary 804 of a surgical cavity 806 in an organ 807 of a patient, largely according to the flowchart of FIG. 9. The electrode array 802 couples through an electrode array 814 connector to an electronics module 810 containing signal conditioning circuitry 812 as above described and as illustrated in the schematic drawings of FIGS. 10 and 11. Module 810 contains a force sensor 816 configured to measure a force or pressure applied through module 810 to force the electrode array 802 of the probe against tissue of the boundary 804 of the surgical cavity. Module 810 is attached to a flexible cable 818 that transmits the signal to the data acquisition system 826 where they are digitized and transmitted to processor 828, processor 828 is configured to use the acquired impedance data from electronics module 810 and the data acquisition system 826 to perform electrical impedance spectroscopy, tomography, mapping, and in some embodiments classification of tissue of the boundary 804 of surgical cavity 806 and to display a map of electrical impedance of the tissue on an attached display 830.

In some embodiments, module 810 contains an optional tracker receiver 822 and is used in a room equipped with a tracker unit 824. In other embodiments, flexible cable 818 is supplemented with a rigid shaft coupling electronics module 810 to handle 820 and handle 820 is equipped with a corresponding optional tracker receiver 822A.

In some embodiments, processor 828 is configured to register a pre-surgical image database 832 or photos from a camera, such as one or more frames from a surgeon's video feed to a tracked location of the patient, as determined by a patient tracking device attached to the patient (not shown in FIG. 8), to determine, from a tracked location and orientation of the handle 820, a location of the probe determined, and to display, on the display coupled to the processor, a location of the probe relative to an image of the pre-surgical image database 832 or one or more photos, such as frames from the surgeon's video feed and to overlay the map of electrical impedance or a map of tissue classification on a tomographic image from the pre-surgical image database or on one or more photos from the surgeon's video feed. In some embodiments, processor 828 is configured to overlay an electrical impedance map or a map of tissue classification on an image of the boundary 804 of surgical cavity 806, the image of the boundary 804 being obtained with a camera prior to, or immediately after, performing impedance mapping of the tissue. Further, processor 828 is configured to use force sensor 816 data acquired with the data acquisition system 826 to measure force applied by a user pressing the probe's electrode array 802 against tissue of the boundary 804 of the surgical cavity and to determine whether this force is greater or less than a predetermined optimum force; processor 828 then provides instructions to the user to increase or decrease the force applied by the user.

In using the system, illustrated in FIG. 8, the system operates according to method 900 (FIG. 9)

In embodiments having the optional probe tracking system, a tracker is attached 902 to the probe, and a second tracker is attached 904 to the patient Location of the patient is registered 906 to preoperative imaging, enabling the processor to track position of the probe relative to patient and to the preoperative imaging. The electrode array is also plugged 908 into the electrode array connector on the laparoscopic probe. Surgery then begins with an initial excision 910 of the tumor, leaving a surgical cavity in the tissue. The surgeon then inserts 912 the laparoscopic probe into the surgical and the DAQ measures 914 applied force. Using indications of insufficient, adequate, and excessive force as measured by the force sensor, the surgeon ensures that correct force is applied 916 to the laparoscopic probe

Although the device is described as a laparoscopic probe, the device may be used in both open and laparoscopic surgeries. Further, while the device is described herein as being used for evaluating surgical margins by probing sides of a surgical cavity after a tumor is removed, the device may also be used to evaluate surgical margins by probing the exterior of a tumor immediately after the tumor has been removed from a patient,

To evaluate surgical margins, in an embodiment, the DAQ system operates under control of the processor in a first pass to control the AFE to couple at least one selected driving electrode to the high-resolution waveform generator, and to couple at least one selected driving electrode to signal ground. The DAQ system then provides 922 a sequence of stimulus signals to the VCCS, each of which may comprise a superposition of one, two, three, or more sine waves of different frequencies in the operating range of 100 to 1,000,000 Hz, while the ADCs measure and digitize voltages at each of the, in an embodiment 25, sense electrodes. The digitized voltages at each of the sense electrodes are input to the PC. The PC then controls the DAQ system in a second pass to control the AFE to couple at least one selected driving electrode, the at least one driving electrode including at least one electrode different than in each prior pass, to the high-resolution waveform generator, and to couple at least one driving electrode of the electrode array to signal ground. The DAQ system then provides 922 a sequence of one or more alternating-current (AC) stimulus signals to the VCCS, each stimulus signal being in embodiments a superposition of one, two, three, or more sine waves of different frequencies in the operating range of 100 to 1,000,000 Hz, while the ADCs measure and digitize voltages at each of the sense electrodes. The digitized voltages at each of the sense electrodes are also input to the PC. The selected driving electrodes are changed, and the sequence repeated 924 until all required measurements are taken. Upon completion of a sequence of passes sufficient for mapping electrical impedance at the probe location, the PC processes the digitized voltages at the sense electrodes for all passes to determine tissue impedances and prepare 926 an impedance map; in embodiments the impedance map is prepared and displayed to a using surgeon 928 as a false color image.

Since contact resistance of driving and sense electrodes to tissue are known to depend on applied pressure of the electrode array to tissue, prior to and during the passes of driving signals onto the driving electrodes and measuring and digitizing voltages at the sense electrodes the DAQ measures 914 force applied between electrode array and tissue by reading the force sensor 816 and providing the force readings to the PC. In embodiments, the PC compares measured force to minimum and maximum force thresholds and provides low force, acceptable force, and excessive force indications to the user with the user increasing or decreasing pressure 918 accordingly to ensure pressure is correct 920 when measurements are taken.

If the bioimpedance map shows insufficient surgical margin, the user may withdraw the probe, remove additional tissue, and repeat 930 application of the probe and mapping of the tissue impedances until either adequate surgical margins are achieved or further tissue removal will cause unacceptable patient outcomes.

In some embodiments, the bioimpedance map is processed by a machine-learning-based classifier to help identify remaining tumor. In particular embodiments, this classifier is a support-vector-machines (SVM) classifier with a radial basis function kernel that has been trained to discriminate normal from positive surgical margins and cancerous tissue for organs of the type being operated on. The SVM classifier has been trained on a training set including a variety of malignant, benign, and normal tissues for that organ type.

### Combinations

Applicants expect the disclosed features may be used in multiple combinations, including:

An apparatus designated A for performing bioimpedance measurements on intraoperative boundaries of surgical cavities including a probe comprising an array of electrodes, the array of electrodes comprising a plurality of pick-up electrodes surrounded by a plurality of driving electrodes, each of the driving electrodes having greater area than each of the pick-up electrodes, the array of electrodes adapted to couple electrically to a connector; the connector coupled to an electronics module, the electronics module adapted to be positioned the array of electrodes and to fit through a laparoscopic port, the electronics module comprising a voltage controlled current source, multiplexing for the driving electrodes, a force sensor, and voltage buffers; the electronics module coupled to a data acquisition system; the data acquisition system coupled to a processor; the processor being configured to use the electronics module and electrode array to perform bioimpedance mapping of tissue contacting the electrode array, and to display the bioimpedance mapping tissue contacting the electrode array on a display coupled to the processor.

An apparatus designated AA including the apparatus designated A wherein the array of electrodes couples to the electronics module through a connector.

An apparatus designated AB including the apparatus designated A or AA wherein there are at least 4 driving electrodes.

An apparatus designated ABA including the apparatus designated AB wherein there are at least 8 driving electrodes.

An apparatus designated AC including the apparatus designated A, AA, AB, or ABA wherein there are at least 25 pick-up electrodes.

An apparatus designated AD including the apparatus designated A, AA, AB, ABA, or AC further comprising a force sensor configured to measure a force with which the electrode array is pressed against the tissue contacting the electrode array.

An apparatus designated AE including the apparatus designated A, AA, AB, ABA, AC or AD wherein the processor is further configured to compare the force with which the electrode array is pressed against the tissue against optimum force limits.

An apparatus designated AF including the apparatus designated A, AA, AB, ABA, AC, AD, or AE further comprising a tracker attached to the shaft, and a tracker attachable to a patient within whom the surgical cavity is formed.

An apparatus designated AFA including the apparatus designated AF wherein the processor is configured to register a tracked location of the patient to an image obtained from a medical imaging system, to determine a location of the probe tip from the tracked location of the probe module, and to display, on the display coupled to the processor, a superposition of the bioimpedance mapping on the medical image.

An apparatus designated AG including the apparatus designated A, AA, AB, ABA, AC, AD, AE, AF, or AFA wherein the electronics module and electrode array form a probe configured to fit through a twelve millimeter diameter laparoscopic port.

An apparatus designated AH including the apparatus designated A, AA, AB, ABA, AC, AD, AE, AF, AFA, or AG wherein the electronics module is coupled to the processor through a data acquisition system (DAQ) configured to conduct electrical impedance imaging with alternating current at a plurality of frequencies between 100 and 1000000 hertz.

An apparatus designated AK including the apparatus designated A, AA, AB, ABA, AC, AD, AE, AF, AFA, AG, or AH wherein there are eight driving electrodes and where the electrode array is about one-third inch in diameter.

A method of mapping bioimpedance of tissue designated B comprising:
contacting the tissue with an electrode array of a probe comprising an array of a plurality of pick-up electrodes surrounded by a plurality of driving electrodes, each of the driving electrodes having greater area than each of the sense electrodes, sequentially driving at least one of the plurality of the driving electrodes with an alternating current at a plurality of frequencies between 100 and 1000000 hertz while reading the plurality of sense electrodes through analog to digital converters of an electronics module into a processor; and using readings of the sense electrodes to generate a bioimpedance map of the tissue; where the electrode array comprises at least four driving electrodes; the electrode array is adapted to couple electrically through a connector to the electronics module, the electronics module adapted to be positioned adjacent the connector and to fit through a laparoscopic port, the electronics module comprising a voltage controlled current source, multiplexing for the driving electrodes, a force sensor, and voltage buffers; and the electronics module is coupled through a data acquisition system into the processor.

A method designated BA including the method designated B wherein there are at least 8 driving electrodes.

A method designated BB including the method designated B or BA where there are at least 25 sense electrodes.

A method designated BC including the method designated B, BA, or BB further comprising using the bioimpedance map of tissue to classify the tissue

A method designated BD including the method designated B, BA, BB, or BC where the tissue is an inner surface of a surgical cavity.

A method designated BE including the method designated B, BA, BB, BC, or BD, where the surgical cavity is created during a radical prostatectomy procedure.

A method designated BF including the method designated B, BA, BB, BC, BD, or BE where the tissue is freshly removed from a patient.

Changes may be made in the above methods and systems without departing from the scope hereof. It should thus be noted that the matter contained in the above description or shown in the accompanying drawings should be interpreted as illustrative and not in a limiting sense. Herein, and unless otherwise indicated: (a) the adjective “exemplary” means serving as an example, instance, or illustration, and (b) the phrase “in embodiments” is equivalent to the phrase “in certain embodiments,” and does not refer to all embodiments. The following claims are intended to cover all generic and specific features described herein, as well as all statements of the scope of the present method and system, which, as a matter of language, might be said to fall therebetween.

1. An apparatus for performing bioimpedance measurements on intraoperative boundaries of surgical cavities comprising:
a probe comprising an array of electrodes, the array of electrodes comprising a plurality of pick-up electrodes surrounded by a plurality of driving electrodes, each of the driving electrodes having greater area than each of the pick-up electrodes, the array of electrodes adapted to couple electrically to a connector;
the connector coupled to an electronics module, the electronics module adapted to be positioned the array of electrodes and to fit through a laparoscopic port, the electronics module comprising a voltage controlled current source, multiplexing for the driving electrodes, a force sensor, and voltage buffers;
the electronics module coupled to a data acquisition system;
the data acquisition system coupled to a processor;
the processor being configured to use the electronics module and electrode array to perform bioimpedance mapping of tissue contacting the electrode array, and to display the bioimpedance mapping tissue contacting the electrode array on a display coupled to the processor.

2. The apparatus of claim 1 wherein the array of electrodes couples to the electronics module through a connector.

3. The apparatus of claim 1 wherein there are at least 4 driving electrodes.

4. The apparatus of claim 3 wherein there are at least 8 driving electrodes.

5. The apparatus of claim 3 wherein there are at least 25 pick-up electrodes.

6. The apparatus of claim 1 further comprising a force sensor configured to measure a force with which the electrode array is pressed against the tissue contacting the electrode array.

7. The apparatus of claim 6 wherein the processor is further configured to compare the force with which the electrode array is pressed against the tissue against optimum force limits.

8. The apparatus of claim 1, 2, 3, 4, 5, 6, or 7 further comprising a tracker attached to the shaft, and a tracker attachable to a patient within whom the surgical cavity is formed.

9. The apparatus of claim 8 wherein the processor is configured to register a tracked location of the patient to an image obtained from a medical imaging system, to determine a location of the probe tip from the tracked location of the probe module, and to display, on the display coupled to the processor, a superposition of the bioimpedance mapping on the medical image.

10. The apparatus of claim 7 wherein the electronics module and electrode array form a probe configured to fit through a twelve millimeter diameter laparoscopic port.

11. The apparatus of claim 9 wherein the electronics module is coupled to the processor through a data acquisition system (DAQ) configured to conduct electrical impedance imaging with alternating current at a plurality of frequencies between 100 and 1000000 hertz.

12. The apparatus of claim 9 wherein there are eight driving electrodes and where the electrode array is one-third inch in diameter.

13. A method of mapping bioimpedance of tissue comprising:
contacting the tissue with an electrode array of a probe comprising an array of a plurality of pick-up electrodes surrounded by a plurality of driving electrodes, each of the driving electrodes having greater area than each of the sense electrodes,
sequentially driving at least one of the plurality of the driving electrodes with an alternating current at a plurality of frequencies between 100 and 1000000 hertz while reading the plurality of sense electrodes through analog to digital converters of an electronics module into a processor;
using readings of the sense electrodes to generate a bioimpedance map of the tissue; where the electrode array comprises at least four driving electrodes;
the electrode array is adapted to couple electrically through a connector to the electronics module, the electronics module adapted to be positioned adjacent the connector and to fit through a laparoscopic port, the electronics module comprising a voltage controlled current source, multiplexing for the driving electrodes, a force sensor, and voltage buffers; the electronics module being coupled through a data acquisition system into the processor.

14. The method of claim 13 wherein there are at least 8 driving electrodes.

15. The method of claim 14 where there are at least 25 sense electrodes.

16. The method of claim 13 further comprising using the bioimpedance map of tissue to classify the tissue

17. The method of claim 16 where the tissue is an inner surface of a surgical cavity.

18. The method of claim 17 where the surgical cavity is created during a radical prostatectomy procedure.

19. The method of claim 16 where the tissue is freshly removed from a patient.

20. The apparatus of claim 9 further comprising a force sensor configured to measure a force with which the electrode array is pressed against the tissue contacting the electrode array and wherein the processor is further configured to compare the force with which the electrode array is pressed against the tissue against optimum force limits.
